(19) United States
(12) Patent Application Publication
(10) Pub. No.: US 2004/0180030 A1
(43) Pub. Date: Sep. 16, 2004

(54) Cosmetic compositions comprising at least one alkylamphohydroxyalkylsulphonate amphoteric surfactant and at least one nacreous agent and/or opacifier, and uses thereof

(76) Inventor: Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/739,083

(22) Filed: Dec. 19, 2003

Related U.S. Application Data
(60) Provisional application No. 60/439,467, filed on Jan. 13, 2003.

(30) Foreign Application Priority Data
Dec. 19, 2002 (FR) 02 16194

(51) Int. Cl. A61K 7/075; A61K 7/08

(52) U.S. Cl. 424/70.21

(57) ABSTRACT

The disclosure relates to novel cosmetic compositions comprising, in a cosmetically acceptable medium, at least one ingredient chosen from opacifiers and nacreous agents, and at least one amphoteric surfactant chosen from alkylamphohydroxyalkylsulphonates and salts thereof. The disclosure also relates to methods for imparting cosmetic properties to keratin materials, such as improved disentangling, smoothing, volume, lightness, softness, flexibility and manageability.

The disclosure still further relates to compositions for washing and/or treating keratin materials, such as the hair or the skin.

This application claims benefit of U.S. Provisional Application No. 60/439,467, filed Jan. 13, 2003.

The present disclosure relates to novel cosmetic compositions comprising, in a cosmetically acceptable medium, at least one ingredient chosen from opacifiers and nacreous agents, and at least one amphoteric surfactant chosen from alkylamphohydroxyalkylsulphonates and salts thereof.

Nacreous agents and opacifiers are known in cosmetic compositions, for example detergent compositions such as shampoos, to give these compositions a nacreous appearance, which users may prefer. It has been found that these nacreous agents may not always give the hair satisfactory conditioning properties.

There is thus a need for cosmetic compositions, for instance detergent cosmetic compositions, such as shampoo, which has a nacreous and/or opacified appearance while at the same time affording acceptable cosmetic performance qualities on keratin materials, for example, on the hair and the scalp.

Alkylamphohydroxyalkylsulphonate amphoteric surfactants are known in detergent cosmetic compositions. They have been described, for example, in Patent Application No. WO 99/36054.

However, these known compositions may not always provide satisfactory cosmetic properties.

One aspect of the present disclosure thus relates to detergent cosmetic compositions that impart improved cosmetic properties, for example, in terms of disentangling and smoothing the hair and making it flexible and manageable.

The present inventor has found, surprisingly, that it is possible to formulate cosmetic compositions for treating keratin materials, for instance, shampoos, with a nacreous and/or opacified appearance while at the same time having desired aesthetic and cosmetic properties, by using in these compositions at least one ingredient chosen from nacreous agents and opacifiers combined with at least one amphoteric surfactant chosen from alkylamphohydroxyalkylsulphonates and salts thereof. For example, it has been found that by using at least one alkylamphohydroxyalkylsulphonate surfactant, satisfactory cosmetic properties can be obtained on keratin materials, such as the hair, for example, the disentangling and/or smoothing of the hair can be improved, and/or the hair can be given volume, lightness, softness, flexibility and/or manageability.

Moreover, when the compositions of the present disclosure are applied to the skin, for example, in the form of bubble baths and shower gels, they afford an improvement in the softness of the skin.

Thus, one aspect of the present disclosure relates to novel cosmetic compositions, comprising, in a cosmetically acceptable medium, at least one amphoteric surfactant chosen from alkylamphohydroxyalkylsulphonates and salts thereof, and at least one ingredient chosen from nacreous agents and opacifiers.

Another aspect of the present disclosure is the use of at least one amphoteric surfactant chosen from alkylamphohydroxyalkylsulphonates and salts thereof in, or for the manufacture of, cosmetic compositions comprising at least one ingredient chosen from nacreous agents and opacifiers.

Still another aspect of the present invention relates to a method for treating keratin materials, such as the hair, comprising applying cosmetic compositions as disclosed herein to the keratin materials.

Yet another aspect of the present disclosure is also the use of a composition as disclosed herein to improve the disentangling and/or smoothing of the hair, and/or to give the hair volume, lightness, softness, flexibility and/or manageability.

As disclosed herein, the term “keratin materials” means the hair, the eyelashes, the eyebrows, the skin, the nails, mucous membranes and/or the scalp, for example, the hair.

The various aspects of the present disclosure will now be described in detail. All the meanings and definitions of the compounds used in the present disclosure given below are valid for all the aspects of the disclosure.

The amphoteric surfactants chosen from alkylamphohydroxyalkylsulphonates may be of formula (I) below:

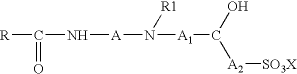

wherein:

R is chosen from saturated and unsaturated, linear and branched hydrocarbon-based radicals comprising from 5 to 29 carbon atoms,

for example, R may be chosen from mono- and polyunsaturated alkyl and alkenyl radicals comprising from 5 to 29 carbon atoms, such as from 7 to 22 carbon atoms, and for instance, from 9 to 17 carbon atoms,

R1 is chosen from C₁-C₄ hydroxyalkyl radicals, such as hydroxyethyl,

A, A₁ and A₂, which may be identical or different, are chosen from linear and branched C₁-C₁₀, for example, C₁-C₃, divalent alkylene radicals,

X is chosen from hydrogen, and mineral and organic cations such as: those of an alkali metal (for example Na⁺ and K⁺), those of an alkaline-earth metal (Mg²⁺ and Ca²⁺), an NH₄⁺ ion, and ammonium ions derived from basic amino acids and/or from amino alcohols.

For example, according to one aspect of the present disclosure, the at least one amphoteric surfactant is chosen from alkylamphohydroxyalkylsulphonates of formula (I) wherein R is chosen from saturated, linear and branched alkyl-radicals comprising from 7 to 29 carbon atoms, such as from 7 to 22 carbon atoms.

When X is an ammonium ion derived from an alkanolamine, the alkanolamine may be chosen from monoethanolamine, diethanolamine, triethanolamine and 3-amino-1,2-propanediol. When X is an ammonium ion derived from an amine, the amine may be chosen from basic amino acids, such as lysine, arginine, sarcosine, ornithine and citrulline.

For example, A may be the same as A₂ and both may be —CH₂CH₂—.

As a further example, A₁ may be —CH₂—.

Also for example, X may be Na⁺.

Among the surfactants of formula (I), non-limiting mention may be made of: cocoyl amphohydroxypropyl sulphonate salts, for example, the sodium salt, such as the product sold under the name MIRANOL CSE by the company Rhodia Chimie, and palmitoyl amphohydroxypropyl sulphonate salts, for example the sodium salt, such as the product sold under the name MIRANOL CS by the company Rhodia Chimie.

As disclosed herein, the at least one amphoteric surfactant chosen from alkylamphohydroxyalkylsulphonates and salts thereof may be present in the composition in an amount ranging from 0.1% to 30% by weight, such as from 1% to 20% by weight, for example, from 1.5% to 15% by weight, relative to the total weight of the final composition.

The at least one ingredient chosen from nacreous agents and opacifiers as disclosed herein, may be chosen from:

i) esters of polyols comprising at least two carbon atoms and of long-chain fatty acids, for example C₁₀-C₃₀ fatty acids, and such as C₁₆-C₂₂, fatty acids; wherein these compounds may optionally be oxyethylenated with 2 to 15 mol of ethylene oxide. The polyols may comprise, for example, from 2 to 4 carbon atoms. Among these compounds, non-limiting mention may be made of: polyethylene glycol monostearates and distearates, for example, ethylene glycol monostearate and ethylene glycol distearate.

ii) long-chain, for example C₁₀-C₃₀, such as C₁₆-C₂₂, fatty acid alkanolamides, such as stearic monoethanolamide, stearic diethanolamide, stearic monoisopropanolamide and stearic monoethanolamide stearate;

iii) esters of long-chain, such as C₁₀-C₃₀, monoalcohols, and of long-chain, such as C₁₀-C₃₀, fatty acids, such as cetyl palmitate;

iv) ethers of long-chain fatty alcohols that are solid at a temperature of less than or equal to approximately 30° C., such as, for example, the dialkyl ethers of formula (II):

R—O—R′ (II)

wherein: R and R′, which may be identical or different, are chosen from saturated and unsaturated, linear and branched alkyl radicals comprising from 10 to 30 carbon atoms, such as from 14 to 24 carbon atoms, and wherein R and R′ are chosen such that the compound of formula (I) is solid at a temperature of less than or equal to approximately 30° C. For example, R and R′ may be a stearyl radical. These ethers can be prepared, for example, according to the process described in Patent Application No. DE 41 27 230. One example of a distearyl ether useful in the composition disclosed herein is sold under the name CUTINA STE by the company Henkel;

v) long-chain (such as C₁₀-C₃₀) esters of long-chain (such as C₁₀-C₃₀) alkanolamides, such as stearamide diethanolamide distearate and stearamide monoethanolamide stearate;

vi) single-chain fatty alcohols comprising at least 16 carbon atoms, for example, at least 20 carbon atoms, such as behenyl alcohol;

vii) C₁₀-C₃₀ long-chain amine oxides, such as (C₁₀-C₃₀)alkyldimethylamine oxides, for example, stearyldimethylamine oxide;

viii) N,N-dihydrocarbyl(C₁₀-C₃₀)amido, for instance (C₁₂-C₂₂)amidobenzoic acids and salts thereof, for example, N,N-di(C₁₆-C₁₈)amidobenzoic acid sold by the company Stepan Company; and

ix) alcohols comprising from 27 to 48 carbon atoms and comprising one or two ether and/or thioether and/or sulphoxide groups, chosen from those of formula (III):

Rₐ—X—[C₂H₃(OH)]—CH₂—Y—R_{b} (III)

wherein

Rₐ and R_{b}, which may be identical or different, are chosen from linear C₁₂ to C₂₄ groups;

X is chosen from an oxygen atom, a sulphur atom, and sulphoxide and methylene groups;

Y is chosen from an oxygen atom, a sulphur atom, and sulphoxide and methylene groups;

and wherein the sum of the number of carbon atoms present in the groups Rₐ and R_{b} has a value ranging from 24 to 44, for example, from 28 to 40, inclusive;

with the proviso that when one of X or Y is sulphoxide, the other of Y or X is not sulphur.

For example, in one aspect of the present disclosure, X may be oxygen, Y may be methylene, and Rₐ and R_{b} may be chosen from radicals comprising 12 to 22 carbon atoms, wherein it is possible for the compounds of formula (III) to be prepared according to Patent No. EP 457 688. As a further example, a compound of formula (III) useful herein is 1-(hexadecyloxy)-2-octadecanol;

x) coated and uncoated titanium oxides, micas and titanium micas; and

xi) cyclodextrins.

In one aspect of the present disclosure, the nacreous agents and opacifiers may be chosen from ethylene glycol monostearate and distearate, distearyl ether, behenyl alcohol and 1-(hexadecyloxy)-2-octadecanol.

As disclosed herein, the at least one ingredient chosen from nacreous agents and opacifiers may be present in the composition in an amount ranging from 0.2% to 15% by weight, for example, from 0.5% to 5% by weight, relative to the total weight of the composition.

In another aspect of the present disclosure, the composition may further comprise at least one cationic polymer.

As used herein, the term “cationic polymer” means any polymer comprising cationic groups and/or groups that may be ionizable into cationic groups. The amphoteric polymers are thus included in this family of compounds.

The cationic polymers that may be used in accordance with the present disclosure may be chosen from any of those already known per se as improving the cosmetic properties of hair treated with detergent compositions, for example, the cationic polymers described in Patent Application EP-A-0 337 354 and French Patent Applications FR-A-2 270 846, 2 383 660, 2 598 611, 2 470 596 and 2 519 863.

For example, the cationic polymers may be chosen from those comprising units comprising primary, secondary, tertiary and/or quaternary amine groups that either may form part of the main polymer chain or may be borne by a side substituent directly attached thereto.

The cationic polymers as disclosed herein may have a number-average molecular mass ranging from 500 to 5×10⁶, for example, ranging from 10³ to 3×10⁶.

Among the cationic polymers as disclosed herein, non-limiting mention made of those chosen from polymers of the polyamine, polyamino amide and polyquaternary ammonium type. These are known products.

The polymers of the polyamine, polyaminoamide and polyquaternary ammonium type that may be used as disclosed herein, include, for example, those described in French Patent Nos. 2 505 348 and 2 542 997.

Among these polymers, non-limiting mention may be made of:

(1) homopolymers and copolymers derived from acrylic and methacrylic esters and amides, and comprising at least one of the units of the following formulae:

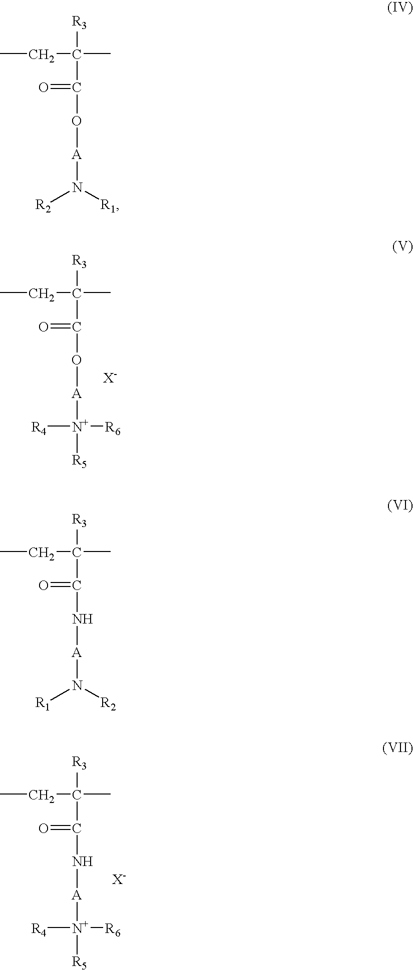

wherein:

R₁ and R₂, which may be identical or different, can be chosen from hydrogen and C₁-C₆ alkyl groups, for example methyl and ethyl;

R₃, which may be identical or different, is chosen from hydrogen and a CH₃ radical;

A, which may be identical or different, is chosen from linear and branched alkyl groups comprising from 1 to 6 carbon atoms, such as from 2 to 3 carbon atoms, and a hydroxyalkyl group comprising from 1 to 4 carbon atoms;

R₄, R₅, and R₆, which may be identical or different, can be chosen from alkyl groups comprising from 1 to 18 carbon atoms and benzyl radicals, for example, alkyl groups comprising from 1 to 6 carbon atoms;

X⁻ is chosen from anions derived from mineral or organic acids, such as a methyl sulphate anion, an ethyl sulphate anion, and halides, such as chloride and bromide.

The copolymers of family (1) can additionally comprise at least one unit derived from comonomers which may be chosen from the family of acrylamides, methacrylamides, diacetone acrylamides, acrylamides and methacrylamides substituted on the nitrogen with a group chosen from lower (C₁-C₄)alkyls, acrylic and methacrylic acids and/or esters thereof, vinyllactams, such as vinylpyrrolidone and vinylcaprolactam, and vinyl esters.

Thus, among the copolymers of family (1), non-limiting mention may be made of:

copolymers of acrylamide and of dimethylaminoethyl methacrylate quaternized with dimethyl sulphate or with a dimethyl halide, such as the product sold under the name HERCOFLOC by the company Hercules,

the copolymers of acrylamide and of methacryloyloxyethyltrimethylammonium chloride described, for example, in Patent Application No. EP-A-080 976 and sold under the name BINA QUAT P 100 by the company Ciba,

the copolymer of acrylamide and of methacryloyloxyethyltrimethylammonium methosulphate sold under the name RETEN by the company Hercules,

quaternized and nonquaternized vinylpyrrolidone/dialkylaminoalkyl acrylate and/or methacrylate copolymers, such as the products sold under the name “Gafquat” by the company ISP, such as, for example, “Gafquat® 734”and “Gafquate® 755”, or alternatively the products known as “Copolymer 845, 958 and 937”. These polymers are described in detail in French Patent Nos. 2 077 143 and 2 393 573,

dimethylaminoethyl methacrylate/vinylcaprolactam/vinylpyrrolidone terpolymers, such as the product sold under the name Gaffix® VC 713 by the company ISP,

vinylpyrrolidone/methacrylamidopropyldimethylamine copolymers sold for example, under the name Styleze® CC 10 by ISP,

quaternized vinylpyrrolidone/dimethylaminopropylmethacrylamide copolymers, such as the product sold under the name “Gafquat® HS 100” by the company ISP.

2) cationic polysaccharides, such as cationic celluloses, cationic starches and cationic galactomannan gums. Non-limiting examples of cationic polysaccharides that may be used include cellulose ethers comprising quaternary ammonium groups, cationic cellulose copolymers and celluloses grafted with a water-soluble quaternary ammonium monomer and cationic galactomannan gums.

The cellulose ethers comprising quaternary ammonium groups, which are described in French Patent No. 1 492 597, and for example, the polymers sold under the names “JR” (JR 400, JR 125, JR 30M) or “LR” (LR 400, LR 30M) by the company Amerchol, may also be used. These polymers are also defined in the CTFA dictionary as hydroxyethylcellulose quaternary ammoniums that have reacted with an epoxide substituted with a trimethylammonium group.

The cationic cellulose copolymers or celluloses grafted with a water-soluble quaternary ammonium monomer are described especially in U.S. Pat. No. 4,131,576. Non-limiting mention may be made of hydroxyalkylcelluloses, for instance hydroxymethyl-, hydroxyethyl- and hydroxypropylcelluloses grafted for example, with a methacryloylethyltrimethylammonium, methacrylamidopropyltrimethylammonium and/or dimethyldiallylammonium salt. Examples of commercial products corresponding to this definition include, for instance, the products sold under the names “Celquat® L 200” and “Celquat® H 100” by the company National Starch.

The cationic galactomannan gums are described, for example, in U.S. Pat. Nos. 3,589,578 and 4,031,307, for instance, guar gums comprising trialkylammonium cationic groups. As disclosed herein, use may be made, for example, of guar gums modified with a salt (e.g., a chloride salt) of 2,3-epoxypropyltrimethylammonium.

Such products are sold for instance, under the trade names Jaguar® C13 S, Jaguar® C 15, Jaguar® C 17 or Jaguar® C162 by the company Rhodia Chimie.

Starches modified with a 2,3-epoxypropyltrimethylammonium salt (e.g., chloride), for instance the product known as Starch hydroxypropyltrimonium chloride according to the INCI nomenclature and sold under the name Sensomer CI-50 from Ondeo, may also be used.

(3) polymers comprising piperazinyl units and divalent alkylene and/or hydroxyalkylene radicals comprising straight or branched chains, which may optionally comprise an entity chosen from oxygen, sulphur, and nitrogen atoms and from aromatic and heterocyclic rings, as well as the oxidation and/or quaternization products of these polymers. Such polymers are described, for example, in French Patent Nos. 2 162 025 and 2 280 361;

(4) water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide and alternatively With an oligomer resulting from the reaction of a difunctional compound that is reactive with a bis-halohydrin, a bis-azetidinium, a bis-haloacyldiamine, a bis-alkyl halide, an epihalohydrin, a diepoxide and a bis-unsaturated derivative; wherein the crosslinking agent is used in an amount ranging from 0.025 to 0.35 mol per amine group of the polyamino amide; these polyamino amides may be alkylated or, if they comprise at least one tertiary amine functions, they may be quaternized. Such polymers are described, for example, in French Patent Nos. 2 252 840 and 2 368 508;

(5) polyaminoamides resulting from the condensation of polyalkylene polyamines with polycarboxylic acids followed by alkylation with difunctional agents. Non-limiting mention may be made, for example, of adipic acid/dialkylaminohydroxyalkyldialkylenetriamine polymers wherein the alkyl radical comprises from 1 to 4 carbon atoms, such as methyl, ethyl and propyl. Such polymers are described for example, in French Patent No. 1 583 363. Among the polyaminoamides, non-limiting mention may further be made of the adipic acid/dimethylaminohydroxypropyl/diethylenetriamine polymers sold under the name “Cartaretine® F, F4 or F8” by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid may range from 0.8:1 to 1.4:1; the polyamino amide resulting therefrom being reacted with epichlorohydrin in a molar ratio of epichlorohydrin to the secondary amine group of the polyamino amide ranging from 0.5:1 to 1.8:1. Such polymers are described for instance, in U.S. Pat. Nos. 3,227,615 and 2,961,347. Polymers of this type are sold for example, under the name “Hercosett® 57” by the company Hercules Inc., as well as under the names “PD 170” or “Delsette® 101” by the company Hercules in the case of the adipic acid/epoxypropyl/diethylenetriamine copolymer.

(7) cationic cyclopolymers for example, alkyldiallylamine or dialkyldiallylammonium, such as the homopolymers or copolymers comprising, as the main constituent of the chain, units corresponding to formulae (VIII) or (IX):

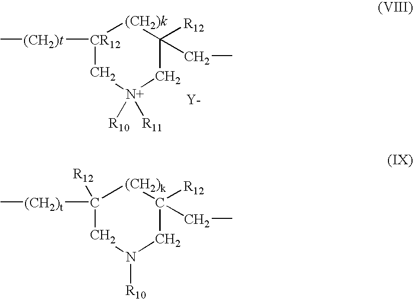

wherein k and t are each equal to 0 or 1, the sum k+t being equal to 1; R₁₂ is chosen from a hydrogen atom or and a methyl radical; R₁₀ and R₁₁, independently of each other, are chosen from an alkyl group having from 1 to 8 carbon atoms, a hydroxyalkyl group wherein the alkyl group, for example, has 1 to 5 carbon atoms, and a lower C₁-C₄ amidoalkyl group, or R₁₀ and R₁₁ can be, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate or phosphate. These polymers are described, for example, in French Patent No. 2 080 759 and in its Certificate of Addition 2 190 406.

For instance, R₁₀ and R₁₁, which may be identical or different, may be chosen from alkyl groups comprising from 1 to 4 carbon atoms.

Among the polymers defined above, non-limiting mention may be made of the dimethyldiallylammonium chloride homopolymer sold under the name “Merquat® 100” by the company Nalco (and its homologues of low weight-average molecular mass) and copolymers of diallyldimethylammonium chloride and of acrylamide, sold under the name “Merquat® 550”.

(8) quaternary diammonium polymers comprising repeating units corresponding to the formula:

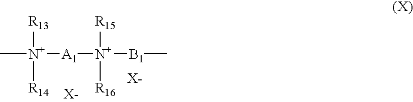

wherein:

R₁₃, R₁₄, R₁₅ and R₁₆, which may be identical or different, are chosen from aliphatic, alicyclic, and arylaliphatic radicals comprising from 1 to 20 carbon atoms, and lower hydroxyalkylaliphatic radicals, or alternatively R₁₃, R₁₄, R₁₅ and R₁₆, together or separately, constitute, with the nitrogen atoms to which they are attached, heterocycles optionally comprising a second hetero atom other than nitrogen, or alternatively R₁₃, R₁₄, R₁₅ and R₁₆ are chosen from linear and branched C₁-C₆ alkyl radicals substituted with a nitrile, ester, acyl or amide group or a group —CO—O—R₁₇-D or —CO—NH—R₁₇-D where R₁₇ is an alkylene and D is a quaternary ammonium group;

A₁ and B₁, which may be identical or different, are chosen from linear and branched, saturated and unsaturated polymethylene groups comprising from 2 to 20 carbon atoms, which may comprise, linked to or intercalated in the main chain, at least one aromatic ring, or at least one atom chosen from oxygen and sulphur atoms, or at least one group chosen from sulphoxide, sulphone, disulphide, amino, alkylamino, hydroxyl, quaternary ammonium, ureido, amide and ester groups, and

X⁻ is an anion derived from an inorganic or organic acid;

wherein A₁, R₁₃ and R₁₅ can form, with the two nitrogen atoms to which they are attached, a piperazine ring. If A₁ is chosen from linear and branched, saturated and unsaturated alkylene and hydroxyalkylene radicals, B₁ can also be a group (CH₂)ₙ—CO-D-OC—(CH₂)ₙ— wherein n ranges from 1 to 100, for example from 1 to 50, and

wherein D is:

a) a glycol residue of formula: —O-Z-O—, wherein Z is chosen from linear and branched hydrocarbon-based radicals and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y, which may be identical or different, are each an integer from 1 to 4, representing a defined and unique degree of polymerization, or any number from 1 to 4 representing an average degree of polymerization;

b) a bis-secondary diamine residue such as a piperazine derivative;

c) a bis-primary diamine residue of formula: —NH—Y—NH—, where Y is chosen from linear and branched hydrocarbon-based radicals, or alternatively the divalent radical

—CH₂—CH₂—S—S—CH₂—CH₂—; and

d) a ureylene group of formula: —NH—CO—NH—.

For example, X⁻ may be chosen from monovalent mineral and organic anions, such as halides, for example, chloride and bromide, sulphates and carboxylates, such as acetate, lactate and citrate.

These polymers may have a number-average molecular mass ranging from 1 000 to 100 000.

Polymers of this type are described for example, in French Patent Nos. 2 320 330, 2 270 846, 2 316 271, 2 336 434 and 2 413 907, and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

In one aspect of the present disclosure, the cationic polymers are chosen from those having repeating units of formula (XI):

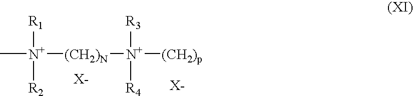

wherein R₁, R₂, R₃ and R₄, which may be identical or different, may be chosen from alkyl and hydroxyalkyl radicals comprising from 1 to 4 carbon atoms, n and p are integers ranging from 2 to 20, and X⁻ is an anion chosen from inorganic and organic acids. In another aspect of the present disclosure., R₁, R₂, R₃ and R₄ are methyl radicals and n=3, p=6 and X=Cl; such a compound is known as hexadimethrine chloride according to the INCI (CTFA) nomenclature.

(9) polyquaternary ammonium polymers comprising units of formula (XII):

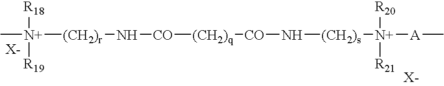

wherein:

R₁₈, R₁₉, R₂₀ and R₂₁, which may be identical or different, are chosen from hydrogen, and methyl, ethyl, propyl, β-hydroxyethyl, β-hydroxypropyl and —CH₂CH₂(OCH₂CH₂)ₚOH radicals,

wherein p is equal to 0 or to an integer ranging from 1 to 6, with the proviso that R₁₈, R₁₉, R₂₀ and R₂₁ do not simultaneously represent a hydrogen atom,

r and s, which may be identical or different, are integers ranging from 1 to 6,

q is an integer ranging from 0 to 34,

X⁻ is an anion, such as a halide,

A is chosen from dihalide radicals, and —CH₂—CH₂—O—CH₂—CH₂—.

Such compounds are described for example in Patent Application No. EP-A-122 324.

Among these compounds, non-limiting mention may be made, for example, of the products “Mirapol® A 15”, “Mirapol® AD1”, “Mirapol® AZ1” and “Mirapol® 175” sold by the company Miranol.

(10) quaternary polymers chosen from vinylpyrrolidones and vinylimidazoles, such as polyquaternium-11, polyquaternium-16 and polyquaternium-44, for instance, the products sold under the names Luviquat® FC 905, FC 550, FC 370 and Luviquat® Care by the company BASF.

(11) polyamines, for instance Polyquart® H sold by Cognis, referenced under the name Polyethylene Glycol (15) Tallow Polyamine in the CTFA dictionary.

(12) Crosslinked and noncrosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example, methylenebisacrylamide. A crosslinked acrylamide/methacryloyloxyethyltrimethylammonium chloride copolymer (20/80 by weight) in the form of a dispersion comprising 50% by weight of the copolymer in mineral oil can also be used. Such a dispersion is sold under the name “Salcare® SC 92” by the company Ciba. A crosslinked methacryloyloxyethyltrimethylammonium chloride homopolymer comprising about. 50% by weight of the homopolymer in mineral oil or in a liquid ester can also be used. Such dispersions are sold under the names “Salcare® SC 95” and “Salcare® SC 96” by the company Ciba.

Other cationic polymers that can be used in the context of the present disclosure include cationic proteins and cationic protein hydrolyzates, polyalkyleneimines, for instance., polyethyleneimines, polymers comprising vinylpyridine or vinylpyridinium units, condensates of polyamines and of epichlorohydrin, quaternary polyureylenes and chitin derivatives.

In one aspect of the present disclosure, the cationic polymers are chosen from cellulose ethers comprising quaternary ammonium groups such as the products sold under the name “JR 400” by the company Amerchol, cationic cyclopolymers, for example, the homopolymers of diallyldimethylammonium salt and the copolymers of diallyldimethylammonium salt and of acrylamide, such as the chlorides, sold under the names “Merquat® 100”, “Merquat® 550” and “Merquate® S” by the company Nalco, and guar gums modified with 2,3-epoxypropyltrimethylammonium chloride, for example sold under the name “Jaguar C13S” by the company Rhodia Chimie.

As disclosed herein, the at least one cationic polymer may be present in the composition in an amount ranging from 0.001% to 20% by weight, such as from 0.01% to 10% by weight, for example, from 0.1% to 3% by weight, relative to the total weight of the final composition.

In another aspect of the present disclosure, the cosmetic compositions as disclosed herein may also comprise at least one silicone.

The at least one silicone that may be used as disclosed herein may be soluble or insoluble in the composition and for example, may be polyorganosiloxanes that are insoluble in the composition. The at least one silicone may be in the form chosen from oils, waxes, resins and gums.

The organopolysiloxanes are defined in greater detail in Walter Noll's “Chemistry and Technology of Silicones” (1968) Academic Press. The at least one silicone may be chosen from volatile and non-volatile silicones.

When the at least one silicone is volatile, then it may be, for example, chosen from those having a boiling point ranging from 60° C. to 260° C.; non-limiting examples of volatile silicones include:

(i) cyclic silicones comprising from 3 to 7, such as from 4 to 5, silicon atoms. For example, octamethylcyclotetrasiloxane sold for instance under the name “Volatile Silicone 7207” by Union Carbide or “Silbione 70045 V 2” by Rhône-Poulenc, decamethylcyclopentasiloxane sold under the name “Volatile Silicone 7158” by Union Carbide, and “Silbione 70045 V 5” by Rhône-Poulenc, and mixtures thereof.

Non-limiting mention may also be made of cyclocopolymers of the dimethylsiloxanes/methylalkylsiloxane type, such as “Volatile Silicone FZ 3109” sold by the company Union Carbide, having the chemical structure:

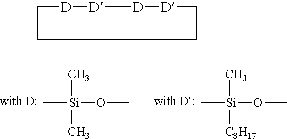

Non-limiting mention may also be made of mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy-111′-bis(2,2,2′,2′,3,3′-hexatrimethylsilyloxy)neopentane;

(ii) linear volatile silicones comprising from 2 to 9 silicon atoms and having a viscosity of less than or equal to 5×10⁶ m²/s at 25° C. An example is decamethyltetrasiloxane sold for instance under the name “SH 200” by the company Toray Silicone. Silicones belonging to this category are also described in the article published in Cosmetics and Toiletries, Vol. 91., Jan. 76, pp. 27-32, Todd & Byers “Volatile Silicone Fluids for Cosmetics”.

When the at least one silicone is chosen from non-volatile silicones, non-limiting examples include: polyalkylsiloxanes, polyarylsiloxanes, polyalkylarylsiloxanes, silicone gums and resins, polyorganosiloxanes modified with organofunctional groups, and mixtures thereof.

Non-volatile silicones may also be chosen from polyalkylsiloxanes, among which non-limiting mention may be made of polydimethylsiloxanes comprising trimethylsilyl end groups having a viscosity ranging from 5×10⁻⁶ to 2.5 m²/s at 25° C., such as from 1×10⁻⁵ to 1 m²/s. The viscosity of the silicones is measured, for example, at 25° C. according to ASTM standard 445 Appendix C.

Further, among the polyalkylsiloxanes, non-limiting mention may be made of the following commercial products:

the Silbione oils of the 47 and 70 047 series or the Mirasil oils sold by Rhône-Poulenc, such as, for example, the oil 70 047 V 500 000;

the oils of the Mirasil series sold by the company Rhône-Poulenc;

the oils of the 200 series from the company Dow Corning, such as, for instance, DC200 with a viscosity of 60 000 cSt;

the Viscasil oils from General Electric and certain oils of the SF series (SF 96, SF 18) from General Electric.

Non-limiting mention may also be made of polydimethylsiloxanes comprising dimethylsilanol end groups (Dimethiconol according to the CTFA name) such as the oils of the 48 series from the company Rhône-Poulenc. In this category of polyalkylsiloxanes, non-limiting mention may also be made of the products sold under the names “Abil Wax 9800 and 9801” by the company Goldschmidt, which are poly(C₁-C₂₀)alkylsiloxanes.

In one aspect of the present disclosure, the polyalkylarylsiloxanes may be chosen from linear and branched polydimethylmethylphenylsiloxanes and polydimethyldiphenylsiloxanes with a viscosity ranging from 1×10⁻⁵ to 5×10⁻² m²/s at 25° C.

Among these polyalkylarylsiloxanes, non-limiting mention may be made, by way of example, of the products sold under the following names:

the Silbione oils of the 70 641 series from Rhône-Poulenc;

the oils of the Rhodorsil 70 633 and 763 series from Rhône-Poulenc;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used as disclosed herein, include, for example, polydiorganosiloxanes having high number-average molecular masses ranging from 200 000 to 1 000 000, used alone or as a mixture in a solvent. The solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecanes.

Non-limiting mention may also be made of the following silicones:

polydimethylsiloxane,

polyd imethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Additional silicones that can be used herein are mixtures such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, such as of a PDMS gum and a PDMS oil, such as the product SF 1236 from the company General Electric. The product SF 1236 is a mixture of an SE 30 gum defined above, having a viscosity of 2 and an SF 96 oil, with a viscosity of 5×10⁻⁶ m²/s. This product may comprise, for example 150/% SE 30 gum and 85% SF 96 oil.

The organopolysiloxane resins that can be used as disclosed herein are chosen from crosslinked siloxane systems comprising the following units:

R₂SiO_{2/2}, R₃SiO_{1/2}, RSiO_{3/2} and SiO_{4/2} wherein R is chosen from hydrocarbon-based groups comprising from 1 to 16 carbon atoms and a phenyl group. For example, the resins may be chosen from those in which R is chosen from C₁-C₄ lower alkyl radicals, such as methyl, and phenyl radicals.

Among the resins, non-limiting mention may be made of the product sold under the name “Dow Corning 593” or resins sold under the names “Silicone Fluid SS 4230 and SS 4267” by the company General Electric, which are silicones of dimethyl/trimethyl siloxane structure.

Non-limiting mention may also be made of the trimethyl siloxysilicate type resins sold for example, under the names X22-4914, X21-5034 and X21-5037 by the company Shin-Etsu.

The organomodified silicones that can be used in accordance with the present disclosure are silicones as defined above and comprising in their structure at least one organofunctional group attached via a hydrocarbon-based radical. Among the Organomodified silicones, non-limiting mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248, and the oils Silwet L 722, L 7500, L 77 and L 711 by the company Union Carbide, and the (C₁₂)alkylmethicone copolyol sold by the company Dow Corning under the name Q2 5200;

substituted and/or unsubstituted amine groups, such as the products sold under the name GP 4 Silicone Fluid and GP 7100 by the company Genesee, and the products sold under the names Q2 8220 and Dow Corning 929 or 939 by the company Dow Corning. The substituted amine groups may be, for example, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax 2428, 2434 and 2440 by the company Goldschmidt;

hydroxylated groups such as the polyorganosiloxanes comprising a hydroxyalkyl function, described in French Patent Application FR-A-85/16334;

acyloxyalkyl groups such as, for example, the polyorganosiloxanes described in U.S. Pat. No. 4,957,732;

anionic groups of carboxylic type, such as, for example, in the products described in patent EP 186 507 from the company Chisso Corporation, and/or of alkylcarboxylic type, such as those present in the product X-22-3701 E from the company Shin-Etsu; 2-hydroxyalkyl sulphonate; 2-hydroxyalkyl thiosulphate such as the products sold by the company Goldschmidt under the names “Abil S201” and “Abil S255”;

hydroxyacylamino groups, such as the polyorganosiloxanes described in patent application EP 342 834. Non-limiting mention may be made, for example, of the product Q2-8413 from the company Dow Corning.

In one aspect of the present disclosure, it is also possible to use silicones comprising a polysiloxane portion and a portion comprising a nonsilicone organic chain, wherein one of the two portions constitutes the main chain of the polymer, the other being grafted onto the said main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers may be, for example, anionic or non-ionic.

Such polymers are, for example, copolymers that can be obtained by free-radical polymerization starting with a monomer mixture comprising:

a) 50 to 90% by weight of tert-butyl acrylate;

b) 0 to 40% by weight of acrylic acid;

c) 5 to 40% by weight of silicone macromer of formula:

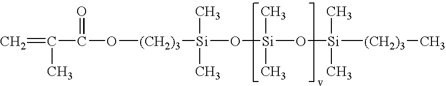

wherein v is an integer ranging from 5 to 700; the weight percentages being calculated relative to the total weight of the monomers.

Other non-limiting examples of grafted silicone polymers include, for example, polydimethylsiloxanes (PDMS) onto which are grafted, via a connecting chain unit of thiopropylene type, mixed polymer units of poly(meth)acrylic acid type and of polyalkyl (meth)acrylate type, and polydimethylsiloxanes (PDMS) onto which are grafted, via a connecting chain unit of thiopropylene type, polymer units of polyisobutyl(meth)acrylate type.

As disclosed herein, the at least one silicone may be used in a form chosen from emulsions, nanoemulsions and microemulsions.

Further non-limiting examples of polyorganosiloxanes as disclosed herein include:

non-volatile silicones chosen from the family of polyalkylsiloxanes comprising trimethylsilyl end groups, such as oils having a viscosity ranging from 0.2 to 2.5 m²/s at 25° C., such as the oils of the DC200 series from Dow Corning, for example, with a viscosity of 60 000 cSt, the Silbione 70047 and 47 series, and for instance, the oil 70 047 V 500 000, sold by the company Rhône-Poulenc, polyalkylsiloxanes comprising dimethylsilanol end groups, such as dimethiconols, and polyalkylarylsiloxanes such as the oil Silbione 70641 V 200 sold by the company Rhône-Poulenc;

the organopolysiloxane resin sold under the name Dow Corning 593;

polysiloxanes comprising amine groups, such as amodimethicones and trimethylsilylamodimethicones.

As disclosed herein, the at least one silicone may be present in the composition in an amount ranging from 0.001% to 20% by weight, such as from 0.01% to 10% by weight, and for instance, from 0.1% to 3% by weight, relative to the total weight of the cosmetic composition.

In yet another aspect of the present disclosure, the cosmetic compositions as disclosed herein may further comprise at least one surfactant other than the alkylamphohydroxyalkylsulphonate amphoteric surfactants disclosed herein, wherein the at least one additional surfactant may be present in the composition in an amount ranging from 0.1% to 60% by weight, such as ranging from 3% to 40%, and for instance, ranging from 5% to 30%, by weight relative to the total weight of the composition.

The at least one additional surfactant may be chosen from anionic, amphoteric, non-ionic and cationic surfactants.

The surfactants that are suitable for use herein may include, for example, the following:

(i) Anionic Surfactant(s):

In the context of the present disclosure, their nature is not an important feature. Thus, as non-limiting examples of anionic surfactants that may be used alone and as mixtures, as disclosed herein, mention may be made of: salts (such as alkaline salts, for example sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates, and N-acyltaurates, wherein the alkyl and acyl radicals of the above described compounds may comprise from 8 to 24 carbon atoms, and wherein the aryl radicals of the above described compounds may be chosen from phenyl and benzyl groups. Among the anionic surfactants that can also be used, non-limiting mention may also be made of fatty acid salts, such as the salts of oleic, ricinoleic, palmitic and stearic acids, coconut oil acid and hydrogenated coconut oil acid; acyl lactylates wherein the acyl radical comprises from 8 to 20 carbon atoms. Weakly anionic surfactants can also be used, such as alkyl-D-galactosiduronic acids and their salts, as well as polyoxyalkylenated (C₆-C₂₄)alkyl ether carboxylic acids, polyoxyalkylenated (C₆-C₂₄) alkylaryl ether carboxylic acids, polyoxyalkylenated (C₆-C₂₄)alkylamido ether carboxylic acids and their salts, for example, those comprising from 2 to 50 ethylene oxide groups, and mixtures thereof.

For instance, as disclosed herein, an anionic surfactant may be chosen from sodium, magnesium and ammonium (C₁₂-C₁₄)alkyl sulphates; sodium, magnesium and ammonium (C₁₂-C₁₄)alkyl ether sulphates oxyethylenated with 2.2 mol of ethylene oxide; and sodium cocoyl isethionate. For example, the anionic surfactant may be chosen from alkyl sulphate salts and alkyl ether sulphate salts.

(ii) Nonionic Surfactant(s):

The non-ionic surfactants are, themselves also, compounds that are well known per se (see, for example, “Handbook of Surfactants” by M. R. Porter, published by Blackie & Son (Glasgow and London), 1991, pp. 116-178) and, in the context of the present disclosure, their nature is not important. Thus, non-limiting examples of non-ionic surfactants as disclosed herein, include: polyethoxylated, polypropoxylated and polyglycerolated fatty acids, alkylphenols, α-diols and alcohols having a fatty chain comprising, for example, 8 to 18 carbon atoms, it being possible for the number of ethylene oxide and/or propylene oxide groups to range, for example, from 2 to 50, and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising, for instance, from 1 to 5, such as 1.5 to 4, glycerol groups; oxyethylenated fatty acid esters of sorbitan comprising from 2 to 30 mol of ethylene oxide; fatty acid esters of sucrose; fatty acid esters of polyethylene glycol; alkylpolyglycosides; N-alkylglucamine derivatives; amine oxides, such as (C₁₀-C₁₄)alkylamine oxides and N-acylaminopropylmorpholine oxides. For instance, as disclosed herein, the non-ionic surfactants may be chosen from alkylpolyglycosides.

(iii) Amphoteric Surfactant(s):

Non-limiting examples of the at least one additional amphoteric surfactant include those chosen from: aliphatic secondary and tertiary amine derivatives wherein the aliphatic radical is chosen from linear and branched chains comprising from 8 to 22 carbon atoms and further comprising at least one water-soluble anionic group (for example carboxylate, sulphonate, sulphate, phosphate and phosphonate). Non-limiting mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

For example, among the amine derivatives, non-limiting mention may be made of the products sold under the name MIRANOL, as described in U.S. Pat. Nos. 2,528,378 and 2,781,354 and having the structures:

R₂—CONHCH₂CH₂—N(R₃)(R₄)(CH₂COO—)

wherein:

R₂ is chosen from alkyl radicals derived from the acid R₂—COOH present in hydrolysed coconut oil, and from heptyl, nonyl and undecyl radicals,

R₃ is a β-hydroxyethyl group, and

R₄ is a carboxymethyl group; and

R₅—CONHCH₂CH₂—N(B)(D)

wherein

B is chosen from —CH₂CH₂OX′ groups,

D is chosen from —(CH₂)ₙ—Y′ groups, wherein z is an integer chosen from 1 and 2, X′ is chosen from —CH₂CH₂—COOH groups and hydrogen, Y′ is chosen from —COOH and the —CH₂—CHOH—SO₃H radical, R₅ is chosen from alkyl radicals of an acid R₉—COOH found in coconut oil and in hydrolysed linseed oil, alkyl radicals, such as C₇, C₉, C₁₁ and C₁₃ alkyl radicals, a C₁₇ alkyl radical and its iso form, and an unsaturated C₁₇ radical and R₉ is chosen from saturated and unsaturated, linear and branched alkyl groups having from 8 to 22 carbon atoms, such as from 8 to 16 carbon atoms.

These compounds are classified in the CTFA dictionary, 5^{th} edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of non-limiting example, mention may be made of the cocoamphodiacetate sold under the trade name MIRANOL C2M concentrate by the company Rhodia Chimie.

(iv) Cationic Surfactant(s):

The cationic surfactants may be chosen from:

A) the quaternary ammonium salts of formula (XIII) below:

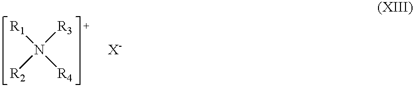

wherein X⁻ is an anion chosen from halides (such as chloride, bromide and iodide) and (C₂-C₆)alkyl sulphates, for example methyl sulphate, phosphates, alkyl or alkylaryl sulphonates, and anions derived from organic acids, such as acetate and lactate, and

i) the radicals R₁, R₂, and R₃, which may be identical or different, are chosen from linear and branched aliphatic radicals comprising from 1 to 4 carbon atoms, and from aromatic radicals such as aryls and alkylaryls. The aliphatic radicals can also comprise hetero atoms such as, for example, oxygen, nitrogen, sulphur and halogens. The aliphatic radicals may be chosen, for example, from alkyl, alkoxy and alkylamide radicals, R₄ may be chosen from linear and branched alkyl radicals comprising from 16 to 30 carbon atoms. For example, the cationic surfactants as disclosed herein may be chosen from behenyltrimethylammonium salts (for example chloride).

ii) the radicals R₁ and R₂, which may be identical or different, may also be chosen from linear and branched aliphatic radicals comprising from 1 to 4 carbon atoms, and from aromatic radicals, such as aryl and alkylaryl. The aliphatic radicals can optionally comprise hetero atoms such as, for example, oxygen, nitrogen, sulphur and halogens. The aliphatic radicals may be chosen, for example, from alkyl, alkoxy, alkylamide and hydroxyalkyl radicals comprising from 1 to 4 carbon atoms;

R₃ and R₄, which may be identical or different, may also be chosen from linear and branched alkyl radicals comprising from 12 to 30 carbon atoms, wherein the radicals comprise least one functional group chosen from ester and amide functional groups. For example, R₃ and R₄ may be chosen from (C₁₂-C₂₂)alkylamido(C₂-C₆)alkyl and (C₁₂-C₂₂)alkylacetate radicals.

As a further example, a cationic surfactant as disclosed herein may be a stearamidopropyldimethyl(myristyl acetate)ammonium salt (for example chloride);

B)—the quaternary ammonium salts of imidazolinium, such as, for example, those of formula (XIV):

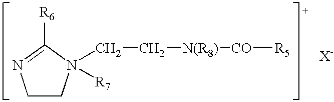

wherein:

R₅ is chosen from alkenyl and alkyl radicals comprising from 8 to 30 carbon atoms, for example fatty acid derivatives of tallow, R₆ is chosen from hydrogen, C₁-C₄ alkyl radicals, and alkenyl and alkyl radicals comprising from 8 to 30 carbon atoms, R₇ is chosen from C₁-C₄ alkyl radicals, R₈ is chosen from hydrogen, and C₁-C₄ alkyl radicals, and X is an anion chosen from halides, phosphates, acetates, lactates, alkyl sulphates, alkyl sulphonates and alkylaryl sulphonates. For example, R₅ and R₆ may be a mixture of alkenyl and alkyl radicals comprising from 12 to 21 carbon atoms, such as, for example, fatty acid derivatives of tallow, R₇ may be methyl and R₈ may be hydrogen.

Such cationic surfactants may be, for example, Quaternium-27 (CTFA 1997) or Quaternium-83 (CTFA 1997), which are sold under the names “Rewoquat” W75, W90, W75PG and W75HPG by the company Witco,

C)—the diquaternary ammonium salts of formula (XV):

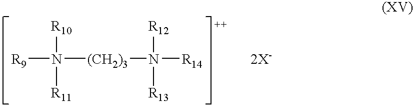

wherein:

R₉ is chosen from aliphatic radicals comprising from 16 to 30 carbon atoms, R₁₀, R₁₁, R₁₂, R₁₃ and R₁₄, which may be identical or different, may be chosen from hydrogen and alkyl radicals comprising from 1 to 4 carbon atoms, and X is an anion chosen from halides, acetates, phosphates, nitrates and methyl sulphates. Such diquaternary ammonium salts may comprise, for example, propanetallowdiammonium dichloride;

D)—the quaternary ammonium salts comprising at least one ester function, of formula (XVI):

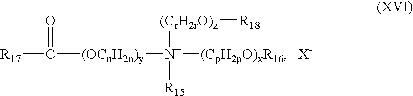

wherein:

R₁₅ is chosen from C₁-C₆ alkyl radicals, C₁-C₆ hydroxyalkyl, and C₁-C₆ dihydroxyalkyl radicals;

R₁₆ is chosen from

radicals of

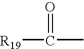

linear and branched, saturated and unsaturated C₁-C₂₂ hydrocarbon-based radicals R₂₀, and a hydrogen atom,

R₁₈ is chosen from:

radicals of

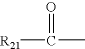

linear and branched, saturated and unsaturated C₁-C₆ hydrocarbon-based radicals R₂₂, and a hydrogen atom,

R₁₇, R₁₉ and R₂₁, which may be identical or different, are chosen from linear and branched, saturated and unsaturated C₇-C₂₁ hydrocarbon-based radicals;

n, p and r, which may be identical or different, are integers ranging from 2 to 6;

y is an integer ranging from 1 to 10;

x and z, which may be identical or different, are integers ranging from 0 to 10;

X⁻ is an anion chosen from simple and complex, organic and inorganic anions;

the sum x+y+z ranges from 1 to 15;

with the provisos that: when x is equal to zero, then R₁₆ denotes R₂₀, and that when z is equal to zero, then R₁₈ denotes R₂₂.

In one aspect of the present disclosure, the ammonium salts of formula (XVI) are used wherein:

R₁₅ is chosen from methyl and ethyl radicals,

x and y are equal to 1;

z is equal to 0 or 1;

n, p and r are equal to 2;

R₁₆ is chosen from:

radicals of

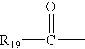

methyl, ethyl and C₁₄-C₂₂ hydrocarbon-based radicals,

a hydrogen atom;

R₁₇, R₁₉ and R₂₁, which may be identical or different, are chosen from linear and branched, saturated and unsaturated C₇-C₂, hydrocarbon-based radicals;

R₁₈ is chosen from:

radicals of

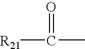

and a hydrogen atom.

Such compounds are sold, for example, under the names Dehyquart by the company Henkel, Stepanquat by the company Stepan, Noxamium by the company Ceca, and Rewoquat WE 18 by the company Rewo-Witco.

Among the quaternary ammonium salts as disclosed herein, non-limiting mention may be made of behenyltrimethylammonium chloride and also stearamidopropyldimethyl(myristyl acetate)ammonium chloride, sold under the name “Ceraphyl 70” by the company Van Dyk, and Quaternium-27 or Quaternium-83 sold by the company Witco.

Mixtures of surfactants, for instance, mixtures of anionic surfactants and mixtures of anionic surfactants and of amphoteric and/or non-ionic surfactants may be used in the compositions as disclosed herein.

The cosmetically acceptable medium may comprise solely water, or a mixture of water and of a cosmetically acceptable solvent such as a C₁-C₄ lower alcohol, for instance ethanol, isopropanol, tert-butanol and n-butanol; and alkylene glycols, for instance propylene glycol, and glycol ethers.

The composition as disclosed herein, for example, may comprise from 50% to 95% by weight of water, relative to the total weight of the composition.

The cosmetic compositions as disclosed herein may have a final pH ranging from 3 to 10. For example, the pH may range from 4 to 8. The pH may be adjusted to the desired value conventionally, by adding a base (such as organic and mineral bases) to the composition, for example, aqueous ammonia, and primary, secondary and tertiary (poly)amines, for instance monoethanolamine, diethanolamine, triethanolamine, isopropanolamine and 1,3-propanediamine, or alternatively by adding an acid, such as a carboxylic acid, for instance citric acid.

The cosmetic compositions as disclosed herein may also comprise at least one additive chosen from thickeners, antidandruff agents, anti-seborrhoeic agents, fragrances, electrolytes, fatty acid esters, preserving agents, silicone and non-silicone sunscreens, vitamins, provitamins, anionic and non-ionic polymers, proteins, protein hydrolysates, 18-methyleicosanoic acid, hydroxy acids, panthenol, plant, animal, mineral and synthetic oils, fluoro and perfluoro oils, natural and synthetic waxes, ceramide compounds, fatty amines, fatty acids and derivatives thereof, fatty alcohols and derivatives thereof, and any other additive conventionally used in cosmetics that does not affect the properties of the compositions as disclosed herein.

The at least one additive may be present in the composition as disclosed herein in an amount ranging from 0 to 20% by weight, relative to the total weight of the composition. The precise amount of each additive may be readily determined by a person of ordinary skill in the art, depending on it's the nature and function of the additive.

The compositions as disclosed herein may be used, for example, for washing and/or treating keratin materials such as the hair, the skin, the eyelashes, the eyebrows, the nails, the lips and/or the scalp, for instance, the hair.

For example, the compositions as disclosed herein may be detergent compositions such as shampoos, shower gels and bubble baths. When the compositions as disclosed herein are detergents, the compositions comprise a washing base, which is generally aqueous.

The at least one surfactant forming the washing base may be chosen, without discrimination, from the anionic, amphoteric and non-ionic surfactants as defined above.

The quantity and quality of the washing base are those that are sufficient to give the final composition satisfactory foaming power and/or detergent power.

As disclosed herein, the washing base can be present in the detergent compositions in an amount ranging from 4% to 50% by weight, such as from 6% to 35% by weight, for example from 8% to 25% by weight, relative to the total weight of the composition.

Another aspect of the present disclosure is a method for treating keratin materials, such as the skin and the hair, comprising applying to the keratin materials a cosmetic composition as defined above, and then optionally rinsing it out with water.

Thus, the method as disclosed herein will allow the maintenance of the hair and the treatment, care, washing and/or the removal of makeup from the skin, the hair and any other keratin material.

In another aspect of the disclosure, the compositions as disclosed herein may also be in a form chosen from rinse-out conditioners, leave-in conditioners, permanent-waving compositions, hair-relaxing compositions, dyeing compositions, bleaching compositions, rinse-out compositions to be applied before or after a process chosen from dyeing, bleaching, permanent-waving and relaxing the hair, and rinse-out compositions to be applied between the two steps of a process chosen from permanent-waving and hair-relaxing.

When the composition as disclosed herein is in the form of a conditioner, optionally a rinse-out conditioner, it may comprise, for example, at least one cationic surfactant, which may be present in the composition in an amount ranging from 0.01% to 10% by weight, such as from 0.5% to 5% by weight, relative to the total weight of the composition.

The compositions as disclosed herein may also be in the form of washing compositions for the skin, for example in the form of bath and/or shower solutions and/or gels, and makeup-removing products.

The compositions as disclosed herein may also be in the form of aqueous or aqueous-alcoholic lotions for skincare and/or haircare.

The cosmetic compositions as disclosed herein may also be in the form of a gel, a milk, a cream, an emulsion, a thickened lotion and a mousse, and may be used for the skin, the nails, the eyelashes, the lips and, for instance, the hair.

The compositions may be packaged in various forms, for example, vaporizers, pump-dispenser bottles, an in aerosol containers to allow the composition to be applied in vaporized form or in the form of a mousse. Such packaging forms are indicated, for example, when it is desired to obtain a spray, a lacquer or a mousse for treating the hair.

Throughout the text hereinabove and hereinbelow, the percentages expressed are on a weight basis.

The present disclosure will now be illustrated more fully with the aid of the examples that follow, which cannot be considered as limiting it to the disclosure aspects described.

In the examples, AM means active material.

### EXAMPLE 1

Two shampoo compositions were prepared, one in accordance with the disclosure (composition A) and the other for comparative purposes (composition B):

| | Disclosure | Comparative |
|---|---|---|
| Composition | (A) | (B) |
| Sodium cocoyl | 12 g AM | — |
| amphohydroxypropylsulphonate, | | |
| as an aqueous solution containing 32% active | | |
| material, sold under the name Miranol | | |
| CSE by the company Rhodia | | |
| Cocoamidopropyl betaine as an aqueous | — | 12 g AM |
| solution containing 30% active material | | |
| Polydimethylsiloxane of viscosity 500 000 | 1 g | 1 g |
| cSt, sold under the name Mirasil | | |
| DM 500 000 by the company Rhodia | | |
| 1-(Hexadecyloxy)-2-octadecanol/cetyl | 2.5 g | 2.5 g |
| alcohol mixture | | |
| Coconut acid monoisopropanolamide | 2 g | 2 g |
| Preserving agents | qs | qs |
| Fragrance | qs | qs |
| Citric acid or sodium hydroxide qs | pH 7 | pH 7 |
| Demineralized water qs | 100 g | 100 g |

A shampoo wash was performed by applying about 6 g of composition A to half a head of premoistened natural hair. The shampoo was worked into a lather and then rinsed out thoroughly with water. The hair was dried with a hairdryer. The same procedure as above was performed with the comparative composition B. Experts compared the half-heads of hair in pairs.

A panel of experts evaluated the appearance of the dried hair, and noted

greater ease of disentangling

a smoother feel

with the composition in accordance with the present disclosure.

Hair treated with composition A was significantly smoother and disentangled more easily than hair treated with composition B.

### EXAMPLES 2 AND 3

The shampoo compositions below were prepared:

| Composition | Example 2 | Example 3 |
|---|---|---|
| Sodium lauryl ether sulphate | 12 g AM | 15.5 g AM |
| (70/30 C12/C14) containing | | |
| 2.2 mol of ethylene oxide | | |
| Sodium cocoyl | 1.8 g AM | 2.2 g AM |
| amphohydroxypropylsulphonate, as an | | |
| aqueous solution containing 32% active | | |
| material, sold under the name Miranol CSE | | |
| by the company Rhodia | | |
| Hydroxyethylcellulose crosslinked with | 0.4 g | — |
| epichlorohydrin and quaternized with | | |
| trimethylamine, sold under the name JR 400 | | |
| by the company Amerchol | | |
| Guar gum modified with 2,3- | — | 0.1 g |
| epoxypropyltrimethylammonium chloride, | | |
| sold under the name Jaguar C13S | | |
| by the company Rhodia | | |
| Polydimethylsiloxane of viscosity 500 000 | 1 g | — |
| cSt, sold under the name Mirasil | | |
| DM 500 000 by the company Rhodia | | |
| Polydimethylsiloxane of viscosity 300 000 | — | 2.7 g |
| cSt, sold under the name DC 200 | | |
| Fluid 300 000 by the company | | |
| Dow Corning | | |
| 1-(Hexadecyloxy)-2-octadecanol/cetyl | 2.5 g | — |
| alcohol mixture | | |
| Distearyl ether | — | 1.5 g |
| Behenyl alcohol | — | 1.5 g |
| Sorbitan monolaurate oxyethylenated with | 8 g | — |
| 4 mol of ethylene oxide, sold under the name | | |
| Tween 21 by the company Uniqema | | |
| Coconut acid monoisopropanolamide | 0.1 g | 1 g |
| Crosslinked polyacrylic acid | 0.2 g | 0.2 g |
| Preserving agents | qs | qs |
| Fragrance | qs | qs |
| Citric acid qs | pH 5.3 | pH 7 |
| Demineralized water qs | 100 g | 100 g |

Hair washed with these compositions was smooth, flexible and manageable.

### EXAMPLES 4 AND 5

The shampoo compositions below were prepared:

| Composition | Example 4 | Example 5 |
|---|---|---|
| Sodium lauryl ether sulphate | 14 g AM | 11.2 g AM |
| (70/30 C12/C14) containing | | |
| 2.2 mol of ethylene oxide | | |
| Sodium cocoyl | 3 g AM | 3 g AM |
| amphohydroxypropylsulphonate, as an | | |
| aqueous solution containing 32% active | | |
| material, sold under the name Miranol CSE | | |
| by the company Rhodia | | |
| Hydroxyethylcellulose crosslinked with | — | 0.8 g |
| epichlorohydrin and quaternized with | | |
| trimethylamine, sold under the name | | |
| JR 400 by the company Amerchol | | |
| Guar gum modified with 2,3- | 0.18 g | — |
| epoxypropyltrimethylammonium chloride, | | |
| sold under the name Jaguar C13S by | | |
| the company Rhodia | | |
| Polydimethylsiloxane of viscosity 60 000 | 2.5 g | — |
| cSt, sold under the name DC 200 | | |
| Fluid 60 000 by the company | | |
| Dow Corning | | |
| 1-(Hexadecyloxy)-2-octadecanol/cetyl | 2.5 g | — |
| alcohol mixture | | |
| Ethylene glycol distearate | — | 2 g |
| Coconut acid monoisopropanolamide | — | 3 g |
| Crosslinked polyacrylic acid | — | 0.2 g |
| Preserving agents | qs | qs |
| Fragrance | qs | qs |
| Citric acid or NaOH qs | pH 7.5 | pH 5.3 |
| Demineralized water qs | 100 g | 100 g |

Hair washed with these compositions was smooth and disentangled easily.

What is claimed is:

1. A cosmetic composition, comprising, in a cosmetically acceptable medium, at least one amphoteric surfactant chosen from alkylamphohydroxyalkylsulphonates and salts thereof and at least one ingredient chosen from nacreous agents and opacifiers.

2. The composition according to claim 1, wherein the at least one amphoteric surfactant is chosen from alkylamphohydroxyalkylsulphonates of formula (I): 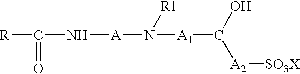
wherein:
R is chosen from saturated and unsaturated, linear and branched hydrocarbon-based radicals comprising from 5 to 29 carbon atoms,
R1 is chosen from C₁-C₄ hydroxyalkyl radicals,
A, A₁ and A₂, which may be identical or different, are chosen from linear and branched C₁-C₁₀ divalent alkylene radicals,
X is chosen from hydrogen, from mineral cations and from organic cations.

3. The composition according to claim 2, wherein R is chosen from mono- and polyunsaturated alkyl and alkenyl radicals comprising from 5 to 29 carbon atoms.

4. The composition according to claim 3, wherein R is chosen from mono- and polyunsaturated alkyl and alkenyl radicals comprising from 7 to 22 carbon atoms.

5. The composition according to claim 4, wherein R is chosen from mono- and polyunsaturated alkyl and alkenyl radicals comprising from 9 to 17 carbon atoms.

6. The composition according to claim 2, wherein R1 is hydroxyethyl.

7. The composition according to claim 2, wherein A, A₁ and A₂, which may be identical or different, are chosen from C₁-C₃-divalent alkylene radicals.

8. The composition according to claim 7, wherein A and A2 are both chosen from —CH₂CH₂—.

9. The composition according to claim 7, wherein A₁ is —CH₂—.

10. The composition according to claim 2, wherein the mineral and organic cations are chosen from alkali metals), alkaline-earth metals, an NH₄⁺ ion, and ammonium ions derived from basic amino acids and/or from amino alcohols.

11. The composition according to claim 10, wherein the alkali metals are chosen from Na⁺ and K⁺.

12. The composition according to claim 10, wherein the alkaline-earth metals are chosen from Mg²⁺ and Ca²⁺.

13. The composition according to claim 1, wherein the at least one amphoteric surfactant is chosen from cocoyl amphohydroxypropyl sulphonate salts and palmitoyl amphohydroxypropyl sulphonate salts.

14. The composition according to claim 1, wherein the at least one amphoteric surfactant is present in the composition in an amount ranging from 0.1% to 30% by weight relative to the total weight of the composition.

15. The composition according to claim 14, wherein the at least one amphoteric surfactant is present in the composition in an amount ranging from 1% to 20% by weight, relative to the total weight of the composition.

16. The composition according to claim 15, wherein the at least one amphoteric surfactant is present in the composition in an amount ranging from 1.5% to 15% by weight, relative to the total weight of the composition.

17. The composition according to claim 1, wherein the nacreous agents and opacifiers are chosen from:
i) esters of polyols comprising at least two carbon atoms and of C₁₀-C₃₀ long-chain fatty acids;
ii) long-chain C₁₀-C₃₀ fatty acid alkanolamides;
iii) esters of long-chain monoalcohols and of C₁₀-C₃₀ long-chain fatty acids;
iv) ethers of C₁₀-C₃₀ long-chain fatty alcohols;
v) long-chain esters of C₁₀-C₃₀ long-chain alkanolamides;
vi) single-chain fatty alcohols comprising at least 16 carbon atoms;
vii) C₁₀-C₃₀ long-chain amine oxides;
viii) N,N-dihydrocarbyl(C₁₀-C₃₀)amidobenzoic acids and salts thereof;
ix) alcohols comprising from 27 to 48 carbon atoms, and comprising one or two ether and/or thioether and/or sulphoxide groups; and
x) coated and uncoated titanium oxides, micas and titanium micas; and
xi) cyclodextrins.

18. The composition according to claim 1, wherein the opacifiers and nacreous agents are chosen from ethylene glycol monostearate, ethylene glycol distearate, distearyl ether, behenyl alcohol and 1-(hexadecyloxy)-2-octadecanol.

19. The composition according to claim 1, wherein the at least one ingredient chosen from opacifiers and nacreous agents is present in the composition in an amount ranging from 0.2% to 15% by weight, relative to the total weight of the final composition.

20. The composition according to claim 19, wherein the at least one ingredient chosen from opacifiers and nacreous agents is present in the composition in an amount ranging from 0.5% to 5% by weight, relative to the total weight of the final composition.

21. The composition according to claim 1, further comprising at least one cationic polymer.

22. The composition according to claim 21, wherein the at least one cationic polymer is chosen from those comprising units comprising primary, secondary, tertiary and/or quaternary amine groups that either form part of the main polymer chain or are borne by a side substituent directly attached thereto.

23. The composition according to claim 21, wherein the at least one cationic polymer is chosen from cellulose ethers comprising quaternary ammonium groups, homopolymers of diallyidimethylammonium salt and copolymers of diallyldimethylammonium salt and of acrylamide, and guar gums modified with 2,3-epoxypropyltrimethylammonium chloride.

24. The composition according to claim 21, wherein the at least one cationic polymer is present in the composition in an amount ranging from 0.001% to 20% by weight, relative to the total weight of the composition.

25. The composition according to claim 24, wherein the at least one cationic polymer is present in the composition in an amount ranging from 0.01% to 10% by weight, relative to the total weight of the composition.

26. The composition according to claim 1, further comprising at least one silicone.

27. The composition according to claim 26, wherein the at least one silicone is chosen from non-volatile polyorganosiloxanes chosen from polyalkylsiloxanes, polyarylsiloxanes, polyalkylarylsiloxanes, silicone gums and resins, and polyorganosiloxanes modified with organofunctional groups.

28. The composition according to claim 27, wherein the at least one silicone is chosen from polyalkylsiloxanes comprising trimethylsilyl end groups, polyalkylsiloxanes comprising dimethylsilanol end groups, polyalkylarylsiloxanes and aminosilicones.

29. The composition according to claim 26, wherein the at least one silicone is present in the composition in an amount ranging from 0.001% to 20% by weight, relative to the total weight of the composition.

30. The composition according to claim 29, wherein the at least one silicone is present in the composition in an amount ranging from 0.01% to 10% by weight, relative to the total weight of the composition.

31. The composition according to claim 1, further comprising at least one additional surfactant chosen from anionic, nonionic, amphoteric and cationic surfactants.

32. The composition according to claim 31, wherein the at least one additional surfactant is present in the composition in an amount ranging from 0.1% to 60% by weight, relative to the total weight of the composition.

33. The composition according to claim 32, wherein the at least one additional surfactant is present in the composition in an amount ranging from 3% to 40% by weight, relative to the total weight of the composition.

34. The composition according to claim 33, wherein the at least one additional surfactant is present in the composition in an amount ranging from 5% to 30% by weight, relative to the total weight of the composition.

35. The composition according to claim 1, further comprising at least one adjuvant chosen from thickeners, antidandruff agents, anti-seborrhoeic agents, fragrances, electrolytes, fatty acid esters, preserving agents, silicone and non-silicone sunscreens, vitamins, provitamins, anionic and nonionic polymers, proteins, protein hydrolyzates, 18-methyleicosanoic acid, hydroxy acids, panthenol, plant, animal, mineral and synthetic oils, fluoro and perfluoro oils, natural and synthetic waxes, compounds of ceramide type, fatty amines, fatty acids and derivatives thereof, and fatty alcohols and derivatives thereof.

36. The composition according to claim 1, wherein the composition is in a form chosen from a shampoo, a conditioner, a permanent-waving composition, a hair-relaxing composition, a hair dyeing composition, a hair bleaching composition, a rinse-out composition to be applied between the two steps of a permanent-waving or hair-relaxing operation, and a washing composition for the body.

37. A method for imparting to the hair at least one property chosen from improved disentangling, smoothing, volume, lightness, softness, flexibility and manageability, comprising applying to the hair a composition comprising, in a cosmetically acceptable medium, at least one amphoteric surfactant chosen from alkylamphohydroxyalkylsulphonates and salts thereof and at least one ingredient chosen from nacreous agents and opacifiers.

38. A cosmetic composition for imparting to the hair at least one property chosen from improved disentangling, smoothing, volume, lightness, softness, flexibility and manageability, comprising, in a cosmetically acceptable medium, at least one amphoteric surfactant chosen from alkylamphohydroxyalkylsulphonates and salts thereof and at least one ingredient chosen from nacreous agents and opacifiers, in a combined effective amount to impart the at least one property.

39. A method for treating keratin materials, comprising applying to the keratin materials a cosmetic composition comprising, in a cosmetically acceptable medium, at least one amphoteric surfactant chosen from alkylamphohydroxyalkylsulphonates and salts thereof and at least one ingredient chosen from nacreous agents and opacifiers and then optionally rinsing the composition out.

40. The method according to claim 38, wherein the keratin materials are hair.

41. A method for imparting softness to skin, comprising applying to the skin a composition comprising, in a cosmetically acceptable medium, at least one amphoteric surfactant chosen from alkylamphohydroxyalkylsulphonates and salts thereof and at least one ingredient chosen from nacreous agents and opacifiers.
